(19) United States
(12) Patent Application Publication
(10) Pub. No.: US 2002/0042057 A1
(43) Pub. Date: Apr. 11, 2002

(54) MLP-gene, nucleic acids, polypeptides and use thereof

(76) Inventor: Ralph Knoell, Friedberg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/773,926

(22) Filed: Feb. 2, 2001

Related U.S. Application Data
(60) Provisional application No. 60/181,928, filed on Feb. 11, 2000.

(30) Foreign Application Priority Data
Feb. 3, 2000 (DE) 10004857.9

(51) Int. Cl. C12Q 1/68; C07H 21/04; C12N 9/00

(52) U.S. Cl. 435/6; 536/23.2; 435/183

(57) ABSTRACT

This invention relates to mutated MLP-sequences, whereby the mutation is carried out at base no. 10 of exon 2 of the translated sequence or at the third position of codon 112 in exon 4 of the translated sequence. In addition, the invention relates to a muscle-specific promoter and uses thereof.

### DESCRIPTION

This invention relates to nucleic acids that code for MLP, a thus coded polypeptide, probes for these nucleic acids, process for detecting and/or screening myocardial diseases, kits for detecting nucleic acids and polypeptide, regulatory nucleic acids, vectors that comprise the latter, cells that comprise the latter and medications that comprise the latter.

The dilatative cardiomyopathy (DCM) is a myocardial disease of unclear origin, which coincides with reduced contractility (systolic disruption of ventricular function) and accompanying disrupted relaxation of the left and/or right ventricle (diastolic disruption of ventricular function). A cardiomegaly with dilatation of both ventricles, reduced emission fraction and high end-diastolic volume is typical. The cardiac output is reduced (forward failure), and it results in the reflux of blood from the heart (backward failure) (Isselbacher, K. et al; Harrison's Principles of Internal Medicine (McGraw Hill, New York, 1994)).

In Western industrialized countries, the incidence of DCM is about 5/100,000 residents/year. It thus represents the most common primary cardiomyopathy. In more recent studies, a family cluster was described in about 30% (Towbin, The Role of Cytoskeletal Proteins in Cardiomyopathies 10, 13-139 (1998)). The costs that accumulate with this disease are, for example, 10-40 billion U.S. dollars in the United States of America, i.e., extrapolated, 5 to 20 billion DM should accumulate in Germany.

The etiology of the disease is still unclear, however. In about 30% of the cases, enteroviral DNAs and RNAs (Pauschinger, M. et al.; Circulation 99(7), 889-895 (1999)) or adenoviral DNAs and RNAs (Pauschinger, M. et al.; Circulation 99(10), 1348-1354 (1999)) can be detected in the myocardium. A possible pathomechanism was suggested by Badorff (Badorff, C. et al.; Nature Medicine 5, 320-326 (1999)). A viral origin can therefore be regarded as likely. Moreover, antibodies, for example against beta-receptors, and the adenine nucleotide translocator (ANT) were detected, and thus autoimmune mechanisms were also discussed as a pathogenetic principle (Schultheiss, H. P. et al; Circ Res. 76, 64-72 (1995)). Increasingly, however, even in the DCM, particularly the family clusters tend to suggest a genetic cause. Mutations in the dystrophin gene, e.g., in the Duchenne and Becker type muscular dystrophy (Muntoni, F. et al.; N Engl J Med 329, 921-5 (1993)), which in addition to muscular dystrophy also result in a DCM, are best documented. Moreover, two mutations in the cardial actin gene were described, which in corresponding families result in a DCM (Olsen, T. et al.; Science 280, 750-752 (1998)). Mutation was also found in the meta-vinculin gene, which very probably resulted in a cardiomyopathy in the patients in question (Maedam, M. et al.; Circulation 95, 17-20 (1997)). In addition, in the cardiomyopathy of the Syrian hamster, it was possible to detect causally a mutation in the delta sarcoglycan gene (Nigro, V. et al.; Hum Mol Genet 6, 601-607 (1997)). These genes code all together for proteins of the cytoskeleton, and thus it is assumed that mutations in cytoskeletal proteins result in a DCM, quite in contrast to the proteins of the sarcomere, in which mutations obviously result in a hypertrophic cardiomyopathy (HCM) (Towbin, J. et al.; Nature Medicine 5, 266-267 (1999) and Chen, J. et al.; J Clin Invest 103, 1483-1485 (1999)).

Since the MLP is listed up to the cytoskeletal proteins, the MLP knock out is added, which coincides with a DCM readily in the above-indicated thought model (Arber et al.; Cell 88, 393-403 (1997)).

In 1994, the MLP was described for the first time as a positive regulator of myogenesis with use of a subtractive cloning technique (Arber, S. et al.; Cell 79, 221-31 (1994)) and in 1997, obtained considerable importance for cardiology, as was known (Arber et al.; Cell 88, 393-403 (1997)), that the MLP-knock out in a mouse model coincides with a DCM. This was simultaneously the first genetically engineered organism that marked this clinical picture. In the prior art, however, it is completely unclear up until now whether mutations in this gene also result in a DCM in humans.

The MLP itself belongs to, as the name expresses, the group of LIM proteins. For about 10 years, this group was named after the starting letters of their first 3 members (lin 11, islet 1, mec 3) and has basically three subgroups: 1. The LIM-kinases, i.e., proteins with the LIM motif and kinase activity, 2. LIM homeobox genes, i.e., LIM proteins, which act as transcription factors, as well as 3. “LIM only” proteins, i.e., LIM proteins that carry LIM motifs exclusively. The MLP belongs to the group of “LIM only” proteins (Morgan, M. et al.; Biochem Biophys Res Com 212, 840-846 (1995)).

The protein itself consists of 194 amino acids and forms two LIM-double zinc fingers, followed by a glycine-rich amino acid sequence in each case. The protein acts as a cytoskeletal protein probably because of its localization on the Z-band, where it binds to f-actin structures (Arber, S. et al.; Genes Dev 10, 289-300 (1996)).

The object of this invention is to provide an agent that allows preventative treatment of dilatative cardiomyopathy. The agent is also to allow the detection of the arrangement of an individual for the development of the clinical picture of the dilatative cardiomyopathy. Finally, the agent that is to be provided is to make possible the diagnosis of dilatative cardiomyopathy.

In addition, an object of the invention is to provide a process for detecting myocardial diseases, for screening myocardial diseases and for detecting the arrangement for the development of a myocardial disease, in each case especially the dilatative cardiomyopathy.

Finally, it is an object of this invention to provide a kit that can be used within the framework of the above-mentioned processes.

In another aspect, the object of the invention is to provide a regulatory nucleic acid, which allows the tissue-specific, especially the muscle-specific, expression of nucleic acids.

In addition, it is an object of this invention to provide agents for treating and/or preventing diseases that can be treated or prevented by tissue-specific expression.

The object is achieved according to the invention by a nucleic acid, which codes for an MLP and comprises a sequence according to SEQ ID NO: 1, whereby the sequence has a mutation at base no. 10 of exon 2 of the translated sequence.

In a preferred embodiment, the mutation is an exchange of T for C.

In addition, the object is achieved by a nucleic acid that codes for an MLP and comprises a sequence according to SEQ ID NO: 1, whereby the sequence has a mutation at the third position of codon 112 in exon 4 of the translated sequence.

In a preferred embodiment, the mutation is an exchange of A for G.

In other preferred embodiments of the nucleic acids according to the invention, the nucleic acid comprises only the sequences that code for an amino acid sequence.

In addition, the object according to the invention is achieved by a nucleic acid that codes for an MLP and comprises the sequence of position 58 to 639 of SEQ ID NO: 1, whereby a mutation is present at position 67.

In a preferred embodiment, the mutation is a mutation from T to C.

In addition, the object is achieved by a nucleic acid, which codes for an MLP and which the sequence of position 58 to 639 of SEQ ID NO: 1, whereby a mutation is present at position 393.

In a preferred embodiment, the mutation is a mutation of A to G.

It is to be noted that in connection with the position data given above, the latter refer to the nucleic acid sequence after SEQ ID NO: 1.

In addition, the object is achieved according to the invention by a nucleic acid that codes for MLP, whereby the sequence would correspond to a nucleic acid according to the invention without the degeneration of the genetic code.

Finally, the object according to the invention is achieved by a nucleic acid that codes for MLP, whereby the nucleic acid hybridizes with a nucleic acid according to the invention.

In another aspect, the object according to the invention is achieved by an amino acid sequence, which is coded by a nucleic acid according to the invention or portions thereof.

In a preferred embodiment, the amino acid sequence according to the invention is coded by the sequence(s) of the nucleic acid according to the invention or portions thereof, which comprise(s) one or both of the above-mentioned mutations, i.e., at base no. 10 of exon 2 of the translated sequence or at the third position of codon 112 in exon 4 of the translated sequence.

In another aspect, the object according to the invention is achieved by an MLP that comprises an amino acid sequence, which is coded by a nucleic acid according to the invention or a portion thereof, or in that the MLP comprises an amino acid sequence according to the invention.

In yet another aspect, the object according to the invention is achieved by a probe for a nucleic acid according to the invention, whereby the probe comprises a sequence according to SEQ ID NO: 4.

In a preferred embodiment, the probe is used for the detection and/or amplification of the nucleic acid according to the invention.

In another aspect, the object according to the invention is achieved by a probe for a nucleic acid according to the invention, whereby the probe comprises the sequence after SEQ ID NO: 5.

In a preferred embodiment, the probe is used for the detection and/or the amplification of the nucleic acid according to the invention.

In another aspect, the nucleic acids according to the invention and/or the probes according to the invention are used individually or in combination for diagnosis and/or screening of myocardial diseases.

In a preferred embodiment in this case, the myocardial disease is the dilatative cardiomyopathy.

In another aspect, the object according to the invention is achieved by a process for detecting and/or screening myocardial diseases, whereby a nucleic acid according to the invention and/or a probe according to the invention is used.

In a preferred embodiment in this case, it is provided that the myocardial disease is dilatative cardiomyopathy.

In another aspect, the object according to the invention is achieved by a process for detecting and/or screening myocardial diseases, whereby a sample that comprises a sequence that codes for MLP is digested with a restriction enzyme, and the presence of a mutated sequence that codes for MLP is detected by the occurrence of a restriction enzyme-digestion pattern that is altered relative to the restriction enzyme-digestion pattern of a non-mutated sequence that codes for MLP.

In this case, it is provided in an embodiment that the myocardial disease is a dilatative cardiomyopathy.

In another embodiment of the process according to the invention, the process is an embodiment of the process according to the invention for detecting and/or screening myocardial diseases, whereby a nucleic acid according to the invention and/or a probe according to the invention is used.

In another embodiment of the process according to the invention, the sequence that codes for MLP is amplified before digestion by PCR.

In another embodiment, the detection or screening is aimed at a nucleic acid according to the invention.

In an especially preferred embodiment, the amplification is carried out with a primer, whereby the primer comprises a sequence according to SEQ ID NO: 13 and/or SEQ ID NO: 14.

In an especially preferred embodiment, it is provided that the restriction enzyme digestion is carried out by Nci I.

In a preferred embodiment, it is provided that in the case of a non-mutated sequence, the PCR product a length of-about 308 bp and in the case of a mutation at base no. 10 of exon 2 or a mutation at position 67 of the nucleic acid sequence of SEQ ID NO: 1, whereby the mutation occurs in an exchange of T for C, two PCR products with about 205 and about 103 bp.

In the process according to the invention, it can be provided that the detection or the screening is aimed at a nucleic acid according to the invention, preferably a nucleic acid that has a mutation at the third position of codon No. 112 in exon 4 of the translated sequence of SEQ ID NO: 1.

In the process according to the invention, it is provided in an embodiment that the amplification is carried out with a primer, whereby the primer comprises the sequence according to SEQ ID NO: 15 and/or SEQ ID NO: 16.

In a preferred embodiment, the restriction enzyme-digestion is carried out by Cvi RI.

In the process according to the invention for detecting and/or screening myocardial diseases, especially dilatative cardiomyopathy, in which a probe according to the invention is used, it can be provided that for the detection or the screening of a nucleic acid sequence according to the invention, a hybridization of a sample that is to be studied is carried out with a probe, whereby the probe comprises a sequence according to SEQ ID NO: 4.

In a preferred embodiment in this case, it is provided that the hybridization is carried out at 60° C.

In the process according to the invention for detecting and/or screening myocardial diseases, especially dilatative cardiomyopathy, whereby a probe according to the invention is used, it can be provided that for the detection or the screening of a nucleic acid sequence according to the invention, a hybridization of a sample that is to be studied is carried out with a probe, whereby the probe comprises a sequence according to SEQ ID NO: 5.

In another aspect, the object of the invention is achieved by a process for detecting and/or screening myocardial diseases, whereby an amino acid sequence according to the invention or an MLP according to the invention is detected.

In a preferred embodiment, the myocardial disease is dilatative cardiomyopathy.

In another preferred embodiment, it is provided that the amino acid sequence or the MLP is detected using an antibody.

In a preferred embodiment, it is provided that the antibody is a monoclonal antibody.

In another aspect of the invention, the object is achieved by a kit for detecting a nucleic acid according to the invention, whereby it is provided that the kit comprises at least one nucleic acid according to the invention and/or at least one probe according to the invention.

In another aspect, the object according to the invention is achieved by a kit for detecting an MLP according to the invention or for detecting an amino acid sequence according to the invention that codes for an MLP, whereby the kit comprises at least one antibody against the MLP according to the invention or against an amino acid sequence according to the invention.

In another aspect, the object is achieved by a regulatory nucleic acid sequence that comprises about 500 base pairs, whereby the 500 base pairs are any that are arranged upstream from the first base of the first exon of the human genomic sequence that codes for MLP.

In an embodiment of the regulatory nucleic acid according to the invention, it is provided that the regulatory nucleic acid comprises about 1000 base pairs, whereby the 1000 base pairs are any that are arranged upstream from the first base of the first exon of the human genomic sequence that codes for MLP.

In another aspect, the object is achieved by a regulatory nucleic acid, which can be produced starting from human genomic DANN with use of two primers in a PCR reaction, whereby the upstream primer comprises SEQ ID NO: 9 and the downstream primer comprises SEQ ID NO: 10.

In another aspect, the object is achieved by a regulatory nucleic acid sequence that comprises a sequence according to SEQ ID NO: 8.

In an embodiment of the regulatory nucleic acid according to the invention, it is provided that the regulatory nucleic acid is a promoter.

In another aspect, the object according to the invention is achieved by a vector that comprises a regulatory sequence according to the invention.

In a preferred embodiment, it is provided that the regulatory sequence is contained in the vector pAd CMVssTnI, whereby in this vector, the CMV promoter is replaced by the regulatory sequence according to the invention.

In a preferred embodiment, the vector according to the invention is an adenoviral vector.

In another embodiment, it is provided that the vector according to the invention comprises a coding sequence, which is under the control of the regulatory nucleic acid according to the invention.

In yet another embodiment, it is provided that the coding sequence is selected from the group that comprises hsp70 and the “slow skeletal troponin I.”

In yet another aspect, the object is achieved by a cell that comprises a regulatory nucleic acid according to the invention and/or a vector according to the invention.

In a preferred embodiment of the cell according to the invention, the cell is a eukaryotic cell.

In a more preferred embodiment, the cell is a mammal cell.

In a quite preferred embodiment, the cell is a human cell.

In another aspect, the object according to the invention is achieved by a medication that comprises a regulatory nucleic acid according to the invention, a vector according to the invention and/or a cell according to the invention.

In a preferred embodiment, the medication is used within the framework of gene therapy.

In another preferred embodiment of the medication according to the invention, it is provided that the medication is one for the treatment and/or prevention of cardiovascular diseases.

In a preferred embodiment, it is provided that the cardiovascular diseases are those in which a point mutation in a gene results in a clinical picture.

In a preferred embodiment of the medication according to the invention, it is provided that the point mutation is in genes that code for proteins of the sarcomere, dystrophin and cardial actin.

In quite especially preferred embodiments of the medication according to the invention, it is provided that the cardiovascular disease is selected from the group that comprises the hypertrophic cardiomyopathy, long QT syndrome and dilatative cardiomyopathy.

The invention is based on the surprising finding that genetic causes for the development or arrangement for development of dilatative cardiomyopathy in humans are of decisive importance. This finding is based on the discovery of the inventor that in various exons of the human MLP-gene, variants are found that coincide with dilatative cardiomyopathy.

The now present genomic human sequence of the MLP gene is organized in six exons, whereby all exons have the standard intron-exon transitions, and elements that are characteristic of a promoter were found on the 5′-end. The locations of the individual exons in the human genomic sequence of the MLP-gene can be described as follows:

Exon 1 of 12983-13011 (28 bp in all)

Exon 2 of 22480-22620 (140 bp in all)

Exon 3 of 26653-26823 (170 bp in all)

Exon 4 of 28611-28746 (135 bp in all)

Exon 5 of 29912-30005 (93 bp in all)

Exon 6 of 32211-32923 (712 bp in all)

The entire gene thus extends to about 20,000 bp.

Exon 1 of the general codes only for a 5′-non-translated area; the translated areas are coded in exons 2 to 6. A computer-aided promoter analysis indicated that there is a standard TATA box before the first exon.

A considerable number of patients with family DCM were analyzed, and in this case it was found, surprisingly enough, that point mutations in the coding sequence of the genomic human sequence of the MLP-gene and thus also in the mRNA or the derived cDNA that correspond in this respect occur.

A patient from such a family showed in exon 2 a base-pair exchange (T→C) that results at 4-position in an amino acid exchange of tryptophan for arginine (W4R). The mutation that causes this exchange takes place more precisely at base no. 10 of exon 2 of the translated human genomic sequence for MLP. The position corresponds to position-67 of the sequence according to SEQ ID No. 1. The mutated sequence is referred to hereinafter as SEQ ID No. 2.

The variant in the MLP-gene discovered by the inventor results in a new restriction interface, and thus large patient populations can be analyzed quickly. Starting from this result, an additional patient population whose members suffer from DCM was studied, whereby the exon 2 was amplified by genomic DNA from patients with DCM and digested with the corresponding enzyme. In this way, from a population of 500 patients, six other patients could be detected with the variant. Family trees were then compiled and, if possible, the family members were called in to give blood samples. Based on these studies, two families have thus far been found in whom the disease co-segregates with the genotype.

Without intending to be doctrinaire, the following considerations relating to the above-described variants (mutants) of the nucleic acid sequence that codes for MLP are to be advanced.

The amino acid tryptophan that is present in the wild type belongs to the heterocyclic amino acids; the arginine that is exchanged for this purpose, i.e., present in the mutated nucleic acid, is the most strongly basic amino acid. This is thus a structure-breaking exchange. The amino acid exchange is in a highly preserved area of the protein, whereby not only the tryptophan is highly preserved at this point between the MLPs of various species (human, pig, rat, mouse) but at the same time also the adjacent amino acids at the amino-terminal end of the protein. This applies not only for the MLP itself, but also for the most closely related proteins (CRP1 and CRP2). It can be concluded from this that the aminoterminal area of the protein is required to transport the MLP to its destination in the myocyte (“to dock,” such sequences are the same in many proteins and therefore are omnipresent). If this is so, the altered MLP could no longer be transported to its destination and possibly had no effect biologically. A computer-aided analysis yielded that the aminoterminal 10 amino acids have a significant homology only to MLP, CRP1 and CRP2. After the exchange, W4R no longer exhibited significant homology to a known protein. At the aminoterminal end of the protein, this thus cannot be a common, typical transporter sequence; moreover, variant W4R also produces no homology to known proteins.

From the above explanations, it can be concluded that the variant in exon 2 has a pathogenetically important function with a probability that borders on certainty in the origin of a DCM in corresponding patients.

In addition to the above-described point mutation, another was found that is associated with DCM. In this case, this is a mutation in exon 4 of the genomic human MLP-gene, more specifically a base exchange at the third position of codon 112, whereby an exchange of G for A is carried out. This exchange does not coincide with any change of the primary sequence of the thus coded MLP-gene product. The position in the translated sequence that corresponds to the above-mentioned genomic position, as it is shown in SEQ ID No. 1, is position 393. The mutated sequence is referred to hereinafter as SEQ ID No. 3.

This mutated form of the MLP-gene or its gene product was found in a patient who was suffering from severe dilatative cardiomyopathy and had to undergo a heart transplant. The origin of the disease was unclear. It was possible to show, however, that in this patient, a clearly reduced MLP-MRNA existed and that this patient has a homozygotic base pair exchange of A for G in exon 4 in codon 112 at position 3. It thus seems to be that this variant results in a reduced half-life of mRNA or, e.g., in an MRNA with other properties via an altered differential processing. It can therefore be assumed that the homozygotic base pair exchange in codon 112 also has a pathogenetically important effect.

The nucleic acids disclosed herein are important in many different respects. By the correlation of the disclosed mutated MRL sequences with heart diseases, especially the dilatative cardiomyopathy, studies of biological material thus can be performed to examine whether the sample and thus the individual from whom the sample originates suffers from a heart disease or at least has an inherent risk of suffering from this heart disease because of the genetic makeup. The use of the disclosed nucleic acids both for purposes of prevention and diagnosis thus can be carried out. In this case, in addition to a study that is geared to the individual, a patient population can also be the subject of studies that use the disclosed nucleic acids or have recourse to the latter.

In this case, the sequences that are claimed herein are to comprise not only the sequences according to SEQ ID No. 2 and SEQ ID No. 3, but also all other sequences that can be derived therefrom, as long as the mutations that are specially disclosed herein are still present. In particular, such derived sequences are to be defined below that have still further mutations beyond the disclosed two special mutations, especially also those sequences that still code for an MLP despite these mutations. Such mutations can be additional point mutations, but also a deletion or an addition of a nucleotide or a complete nucleotide sequence. It is also conceivable that several deletions or additions are present in such a sequence. These additions and deletions can be carried out at various points of the sequence. of the nucleic acids that are disclosed herein, those are also comprised that comprise the sequences according to SEQ ID No. 2 or SEQ ID No. 3, whereby portions of any of the two sequences are separated by a nucleotide or a sequence of several nucleotides. An example of such a sequence is the genomic MLP-sequence, which has the above-mentioned mutations, and whose coding areas are separated from one another by intron.

The production of the genomic human MLP sequence is described herein in the examples.

The probes that are disclosed herein are allele-specific oligonucleotides, which are complementary to those areas of the nucleic acids that carry the disclosed mutations. These probes can be used in particular in the polymerase-chain-reaction as primers for the amplification of nucleic acid and allow the detection itself at small sample amounts. The probes can be used either after the polymerase reaction or directly to detect the nucleic acids that exhibit the mutations. For this purpose, the probes can be present in labeled form. These labelings can be, i.a., radiative or florescent markers.

The sequence of the probes or primers for detecting mutation in exon 2 reads:

AGA TGC CAA ACC GGG GCG

and is described herein as SEQ ID No. 4, and for detecting mutation in exon 4:

TTC ACT GCG AAG TTT GGA

and is described herein as SEQ ID No. 5.

In the case of exon 2, the sequence of the probes or primers for detecting the wild-type sequence that corresponds to the respective mutations reads:

AAGA TGC CAA ACT GGG GCG

and is described herein as SEQ ID No. 6, and in the case of exon 4:

TTC ACT GCA AAG TTT GGA

and is described herein as SEQ ID No. 7.

In the process according to the invention, the sample working-up steps that are commonly used for detecting nucleic acids or gene products are performed that are known to experts in the field. As starting material, for example, blood or biopsy material can be used.

If the process according to the invention is aimed at detecting mutation via the production of an additional interface for a restriction enzyme, which is not present in the wild-type sequence, either in the genomic sequence, the MRNA or a CDNA, the nucleic acid fragments that are obtained are generally separated in an agarose gel. Other separating techniques are also conceivable, however, thus, e.g., capillary electrophoresis. If an agarose gel is used, the various nucleic acid fragments are made visible by staining with, for example, ethidium bromide or as a result of a radioactive marker.

If in one of the processes according to the invention the gene product of one of the nucleic acids that is described herein is detected, all methods that are suitable for detecting peptides or proteins can be used for this purpose. A possibility exists in the use of antibodies, especially monoclonal antibodies.

It is obvious to experts that when detecting a peptide or protein that is coded by a nucleic acid that exhibits the mutation in exon 4, in which it results in an exchange at position 3 of codon 112 from A to G, it is necessary to shift to other detection processes that do not focus on the different nature of the gene product because of the mutation, since this mutation is a silent mutation that does not manifest itself in the plane of the primary sequence, i.e., in the amino acid sequence. As noted above, however, in this mutation, particularly in the presence of a homozygotic base pair exchange, i.e., both alleles have this mutation, it results in a considerably reduced MLP-mRNA, so that on the plane of the gene product, the detection of the mutation can be carried out by quantification of the mRNA or the gene product.

The kits according to the invention comprise at least one of the nucleic acids according to the invention, probes or a gene product of the above-mentioned nucleic acids. At least one probe that is specific to one of the two described mutations especially preferably comprises the kit according to the invention. In this case, the probe that corresponds to the wild type can be contained as a negative control. The nucleic acids according to the invention can be contained in the kit as positive and/or negative controls. As an alternative or in addition to the probe, a restriction enzyme can be contained in the kit that cuts in at the interface newly produced by the mutation and thus allows a restriction digestion of the mutated sequence in comparison to the non-mutated sequence and thus results in another pattern of the —cut— nucleic acid, i.e., a restriction length polymorphism. The nucleic acids, probes or gene products can be present in the kit here in a suitable buffer. The kit can also comprise a vehicle according to the invention in a nucleic acid or a polypeptide or protein can be immobilized.

In another aspect, the invention relates to a regulatory nucleic acid, particularly a promoter.

Hereinafter, the terms regulatory nucleic acid, promoter structure and promoter are used synonymously.

In the studies that are performed by the inventor, it was made obvious that a sequence arranged before the first exon of the genomic human MLP-sequence with a length of about 2000 bp for the muscle-specific expression seems to be of considerable importance. In particular, both a TATA box and a CAAT box, which are both important for the expression of the gene, are found before the first exon. Moreover, AP-1 cis-regulatory elements were found, of which it is known that they are important for muscle-specific genes. The described regulatory nucleic acid sequence otherwise results in that the expression of the MLP can be induced by stress. The regulatory nucleic acid sequence thus represents a tissue-specific promoter structure, which in addition can be induced by stress.

It follows from the above-mentioned tissue specificity of the regulatory nucleic acid that the MLP-gene occurs only in the muscle tissue (i.e., striped and smooth muscles); this is thus a muscle-specific promoter. The gene product or the mRNA can be detected relatively simply with various techniques (e.g., Northern Blot, PCR), which can conclude in a strong promoter. The first exon codes only about 30 base pairs of the 5′-non-translated area. Several TATA-box elements lie about 20-30 base pairs before the first exon, thus specifically the areas to which the DNA-dependent RNA-polymerase II must bind to transcribe the gene. Moreover, several transcription initiation sequences lie about 10 to 20 base pairs before the first exon. In addition, several AP-1 sequences are present directly before the first exon. These sequences bind the so-called “leucine zipper” transcription factors, which take up important functions, i.a., in the stress inducibility of genes. The presence of these sequences confirms the stress inducibility of the MLP-gene found by the inventor.

A complete series of AP-1 sequences are found “upstream” in the first 1000 base pairs. A CAAT box is found about 500 base pairs upstream. These elements are generally found 80-120 base pairs before the gene, but an important function of this element in the promoter discovered by the inventor is not ruled out. It must be noted, however, that it also provides promoters, mainly tissue specific promoters without CAAT boxes, thus this is not an absolutely necessary element.

In summary, it can be noted that AP-1 and CAAT sequences are found up to about 1000 base pairs before the first exon. The area of about 1000 base pairs before the first exon thus seems to be of considerable importance for the expression of the gene. As a nuclear region, in any case, the area of about 500 base pairs (thus starting from the CAAT box) can be designated before the first exon.

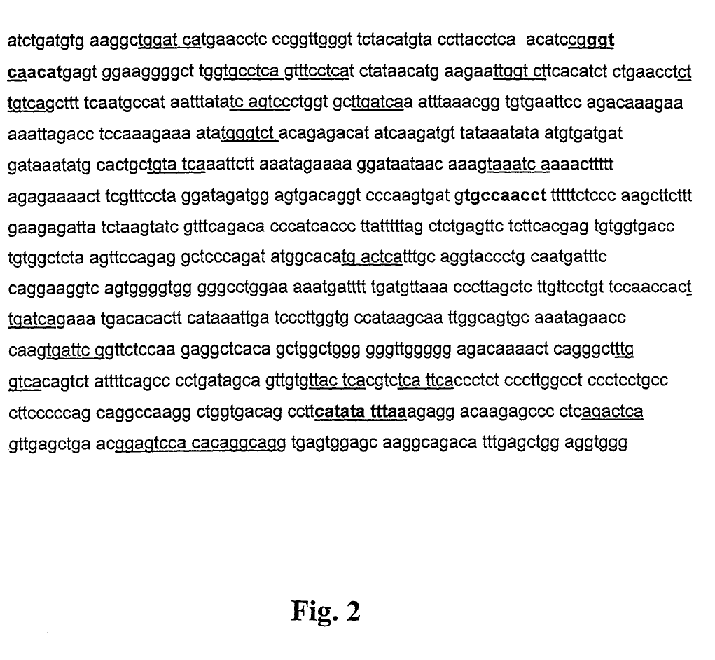

The various elements of the regulatory nucleic acid disclosed herein are depicted in FIG. 2.

The regulatory sequence according to the invention can be depicted in another embodiment also by the sequence according to SEQ ID No. 8.

The production of the regulatory sequence or promoter structure according to the invention can also be carried out in that the corresponding sequence of human, genomic DNA is amplified with two primers with the aid of the polymerase-chain reaction. In this regard, primer gP1 with the sequence

ATC TGA TGT GAA GGC TGG

is used as an “upstream” primer, as it is also indicated in the sequence protocol as SEQ ID No. 9.

As a “downstream” primer, primer gP2 is used with sequence

CCT GCC TGT GTG GAC TCC

as it is also indicated in the sequence protocol as SEQ ID No. 10.

The regulatory sequence according to the invention can also be present integrated in a vector. Such vectors can be both plasmid vectors and viral vectors. Typically, vectors are nucleic acid molecules that are used for —preferably foreign— nucleic acids as vessels or vehicles. In general, vectors carry a replication origin (“origin”) and genetic markers that allow for the vector to be detected in host cells. Moreover, the vector can comprise additional elements that are used in the control of the translation and/or transcription of the nucleic acid that is taken up or contained in the vector.

Both the regulatory sequence according to the invention and the vector according to the invention can be contained in a cell. For this purpose, basically any cell or cell line can be used. The cell can be selected from the group that comprises prokaryotic and eukaryotic cells. Within the group of eukaryotic cells, the cell can in turn be selected from the group that comprises in particular yeast cells, insect cells and mammal cells. In particular, it can be provided in the case of mammal cells that these are primary cells, primary cell cultures or established cell cultures.

For the regulatory sequences and the various biological systems that contain the latter, a considerable number of possible applications are produced that are based on the following considerations as well as the fact that the regulatory nucleic acid disclosed herein allows a tissue-specific and in particular muscle-specific expression of nucleic acid sequences that are under the control of the above-mentioned regulatory sequence.

A considerable number of human diseases can be attributed to genetic defects. A major part of these clinical pictures is in turn triggered by monogenous diseases, i.e., mutations in a single gene. With knowledge of these molecular alterations, a repair of the molecular defect can be performed by gene therapy.

In the cardiovascular system, a number of diseases are known in which point mutations in a single gene lead to serious clinical pictures. These include mutations in the genes, which code proteins of the sarcomere and result in hypertrophic cardiomyopathy (Towbin. The Role of Cytoskeletal Proteins in Cardiomyopathies. 10, 131-139 (1998)), the long QT syndrome (Jervell and Lange Nielson syndrome and Romano-Ward syndrome) (Neyround, N. et al.; Circ Res 84, 290-297 (1999) as well as mutations in dystrophin (Muntni, F. et al.; N Engl J Med 329, 921-5 (1993)) or cardial actin gene (Olsen, T. et al.; Science 280, 750-752 (1998)), which result in dilatative cardiomyopathy. These diseases can be treated causally in principle by administration of the correct gene.

Just recently, various authors reported on the successful administration of adenoviruses in cardiomyocytes in vitro and in vivo (Hajjar, R. et al.; Proc Natl Acad Sci USA 95, 5251-5256 (1998); Donahue, J. et al.; Proc Natl Acad Sci USA 94, 4664-4668 (1997) and Westfall, M. et al.; Proc Natl Acad Sci USA 94, 5444-5449 (1997)). Vectors used up until now, however, have the drawback that they are controlled by a cytomegalovirus (CMV) promoter. They are therefore expressed not only in the myocardium or the muscle tissue, but also in any other body cell. The above-cited clinical pictures pertain only to cardiomyocytes, however. An expression of the gene that is to be transferred into cells other than in cardiomyocytes makes no sense and only increases the danger of a gene therapy, namely the occurrence of further mutations or undesirable interactions within the meaning of immune reactions. The regulatory nucleic acid according to the invention is expressed only in the muscle tissue in contrast to the CMV protomer that is expressed ubiquitously and is therefore extremely well suited for gene therapy in or of muscle tissue based on its strength.

In the production of the regulatory nucleic acid according to the invention, corresponding sequences that can be detected by a restriction enzyme can generally be added at their ends, and particularly if the production is carried out using the two primers described above (e.g., Eco RI or any other enzyme on whose interfaces the cloning is to take place).

In the production of a vector according to the invention, the procedure can be that it is excised from the shuttle vector pAdCMVssTnI, as described in Westfall et al. (Westfall, M. et al.; Proc Natl Acad Sci USA 94, 5444-5449 (1997)), with the corresponding enzyme of the CMV-promoter and is incorporated into the MLP-promoter (i.e., a regulatory nucleic acid according to the invention) in the shuttle-plasmid pAdCMVssTnI. The plasmid pAdMLPssTnI that is now produced can be co-transfixed into HEK 293 cells together with pJM17 via the calcium phosphate method, as described in Westfall et al. (Westfall, M. et al.; Proc Natl Acad Sci USA 94, 5444-5449 (1997)), and an adenovirus with MLP-promoter, which is expressed in a tissue-specific manner, can be obtained by homologous recombination. This virus was expressed as a special case of the so-called “slow skeletal troponin I.” It can, however, be cloned in any desired gene product in a known way for the MLP-promoter in a suitable vector, or a shuttle vector and expressed. For example, the hsp70 gene, which takes up protective functions on the myocardium, could be placed upstream (Yellon, D. et al.; J Mol Cell Card 24, 113-124 (1992)). In this case, only standard cloning methods are necessary, which are known to experts in the field and are described in, for example, Maniatis, Fritsch & Sambrook, Molecular Cloning. Cold Spring Harbor Laboratories, 1989.

In summary, it can be noted that the promoter according to the invention can be cloned for any desired gene product, and the promoter according to the invention thus can bring to expression by tissue specificity with any desired virus or vector system the nucleic acid that is under the control of the promoter. In this respect, the promoter disclosed herein can be used particularly in the case of the above-mentioned and herein-disclosed diseases, including the gene-therapeutic treatment of DCM. In addition to the diseases that are mentioned explicitly herein, all of those diseases can also be treated with use of the regulatory sequence according to the invention, in which there is a need for tissue-specific expression and particularly the muscle-specific expression of a nucleic acid.

The invention is further explained based on the following examples, the figures and the sequence protocol, from which additional features and advantages of the invention can be produced.

Here:

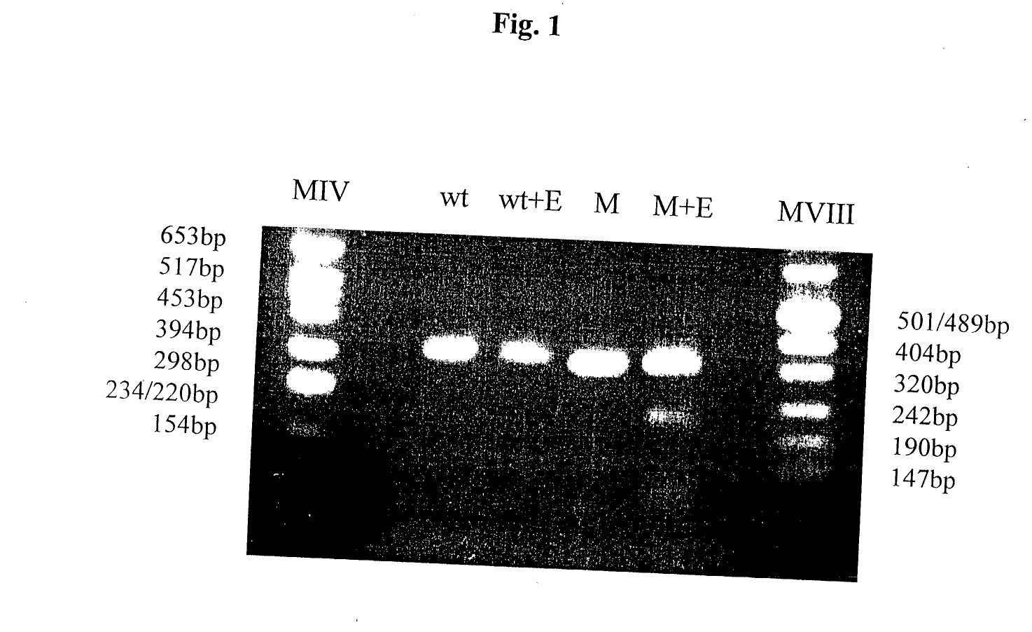

FIG. 1 shows a restriction analysis of exon 2 of the genomic human MLP-gene; and

FIG. 2 shows the sequence of the regulatory nucleic acid according to the invention with the promoter-specific elements.

### EXAMPLES

1. Production of Human MLP-cDNA

Human MLP-cDNA was produced with the aid of the polymerase-chain reaction (=PCR) with use of the two primers KMLP1 and KLMP2.

The sequence of KMLP1 is produced as SEQ ID No. 11 and reads as follows:

GGC AGA CTT GAC CTT GAC CA

The sequence of KMLP2 is produced as SEQ ID No. 12 and reads as follows:

GAG GTG CGC CGT TTC TCA GA

The cDNA that is obtained in such a way as a product of PCR has a length of about 650 bp and contains the entire coding area of the mRNA.

2. Production of the Human Genomic MLP-Sequence

Starting from the cDNA described in Example 1, the human genomic MLP-sequence was produced in that a human genomic gene bank was screened with use of cDNA. For this purpose, the cDNA used as a probe was radiolabeled (Feinberg, A. et al.; Anal Biochem 132, 6-13 (1983)) and hybridized with a gene bank cloned in bacteriophages P1 (Ioannou, P. A. et al.; Nature Genetics 6, 84-89 (1994)). The screening was carried out with use of the common methods known to experts, as described in, e.g., Maniatis, Fritsch & Sambrook, Molecular Cloning, Cold Spring Harbour Laboratories, (1989).

The MLP-gene itself is located on chromosome llpl5.1 (Fung, Y. W. et al.; Genomics 28, 602-603 (1995)).

The clone of the human genomic MLP-sequence that was obtained in such a way was then sequenced using an automatic DNA sequencer. The positive clone comprised a total of 80,000 base pairs and contains the entire human MLP-gene, which is organized in a total of six exons, as already disclosed above.

3. Detection of Mutations in Exon 2 and in Exon 4 of the Human Genomic MLP-Sequence

As determined above, the two mutations in the MLP-sequence found by the inventor are one at base 10 of exon 2 of the translated human genomic MLP-sequence, whereby an exchange of T for C is carried out, and one at the third position of codon 112 of exon 4 of the translated sequence, whereby an exchange of A for G is carried out. The counterparts to both mutations are the mRNA or the cDNA that can be derived therefrom, whereby in the case of the mutation in exon 2, the position of the mutation of base 67 corresponds to mRNA or cDNA, and in the case of the mutation in Example 4, the position of the mutation of base 393 corresponds to the mRNA or the cDNA, as also depicted in SEQ ID No. 1.

3.1 Isolation of the Genomic DNA

Production of the “Burry Coats”

Whole blood is centrifuged for about 10 minutes at 3300 g and at room temperature. After centrifuging, three phases are obtained: the upper clear phase corresponds to the plasma, the thin white phase corresponds to the leukocytes, and the lower red phase corresponds to the erythrocytes. The leukocytes can now be removed easily with a pipette. They correspond to the “buffy coat.”

Isolation of the Genomic DNA

The isolation of the genomic DNA was carried out as follows with the aid of a “Qiagen Blood Kit” of 1997 and a standard “Eppendorf tabletop centrifuge” :

a) About 200 μl of the buffy coat was added to a 1.5 ml Eppendorf vessel. 25 μl of proteinase K and 200 μl of a buffer AL were added to it and vortexed (vigorously shaken, mixed) for 15 seconds.

b) This mixture was heated for 10 minutes in a water bath at 70° C.

c) 210 μl of ethanol (100%) was added to this mixture and vigorously stirred again.

d) A quiagen column was added to a 2 ml Eppendorf vessel, and the mixture from c) was applied to the column. centrifuging was carried out for 1 minute at 6000 g, and the column was added to a new 2 ml Eppendorf vessel. The centrifuged liquid was discarded, the genomic DNA had bonded to the column.

e) 500 μl of buffer AW was added to the column and again centrifuged at 6000 g for 1 minute. The centrifuged liquid was again discarded, and the column was added to a new 2 ml Eppendorf vessel.

f) 500 μl of buffer AW was again added to the column and centrifuged at the highest speed (about 15,000 rpm in an Eppendorf tabletop centrifuge) for 3 minutes. The centrifuged liquid was again discarded.

g) The column was now placed on a 1.5 ml Eppendorf vessel, and 200 μl of pure water, previously heated to 70° C., was added to the column. Incubation was carried out for one minute at room temperature, and centrifuging was carried out at 6000 g for one minute. In the liquid that was centrifuged off, the eluate, the genomic DNA that was used for other purposes was found. The concentration was determined photometrically in another step.

For the polymerase-chain reaction, about 50 mg of the genomic DNA was used.

Amplification of Exon 2 of the Human Genomic MLP-Sequence

For amplification of exon 2, primer GMLP 3 was used with sequence

ACT CTT AGA GAT TGG TTC ACT CC

corresponding to SEQ ID No. 13 and GMLP 4 with the sequence

ACC ACA CTA TGA GAA CCA CTG GC

corresponding to SEQ ID No. 14.

In this case, the following amplification conditions were used:

1. 94° C. for 4 minutes

Then a total of 36 cycles begin with

2. 94° C. for 45 seconds

3. 62° C. for 45 seconds

4. 72° C. for 1 minute.

In connection with the 36 cycles then:

5. 72° C. for 5 minutes.

3.2 Restriction Digestion of Exon 2 of the Human Genomic MLP-Seguence

A portion of the PCR products described and obtained under 3.1 was separated in an agarose gel and analyzed to determine its correct size. In the case of correct sizes, which was generally the case, another portion was digested with restriction enzyme Nci I overnight at 37° C. and separated again with an agarose gel the next morning.

The PCR for amplification of exon 2 yields a PCR product with a size of 308 base pairs. If a base pair exchange of T for C has occurred in codon 4 at position 1, restriction enzyme Nci I is cut, and two fragments are produced with a size of 103 base pairs and 205 base pairs.

In the homozygotic case, no PCR-product with a size of 308 base pairs was found, but rather only the two fragments. In the heterozygotic case, i.e., a healthy allele, i.e., non-mutated relative to the special base, and a mutated allele relative to the special base are present, the PCR product with 308 base pairs additionally is found in the two smaller fragments.

The result of this restriction analysis is depicted in FIG. 1, whereby FIG. 1 shows more precisely the result of a restriction digestion of amplified exon 2 of the human genomic MLP-sequence, which was applied to an agarose gel and was separated. The two respectively external traces of the agarose gel show a molecular weight standard. The trace that is transcribed by “wt” shows exon 2 in its wild type, whereby no restriction enzyme was added to the applied restriction batch. The trace that is transcribed by “wt +E” shows exon 2 in its wild type, whereby restriction enzyme Nci I was added to the applied restriction batch. Because of the absence of the mutations disclosed herein at base 10 of exon 2 in the human genomic wild-type-MLP-sequence, no interface for Nci I is present in exon 2, so that no altered pattern is produced relative to the restriction batch with the wild-type-sequence without a restriction enzyme. The trace that is transcribed by “M” shows a restriction batch that was performed with the mutated form of exon 2 of the human genomic MLP-sequence without a restriction enzyme. Without the addition of the restriction enzyme, no additional strips are found in the gel, so that as is evident from the comparison with the batch referred to as “wt,” the size of the nucleic acid fragment that comprises exon 2 is not altered because of the mutation. If a restriction batch with the exon 2 that is mutated at base 10 is implemented, and restriction enzyme Nci I is added, this results in a cutting of nucleic acid by exon 2 because of the mutation-induced design of an interface for the above-mentioned restriction enzyme and a design of the two nucleic acid fragments mentioned above, as is also shown in FIG. 1 in the trace that is transcribed by “M+E.”

With use of this technique, to date about 500 patients have been examined for the occurrence of the variants discovered by the inventor. Six patients were found with the variants.

3.3 Restriction Digestion of Exon 4 of the Human Genomic MLP-Sequence

For analysis of exon 4, the procedure in principle is just as in exon 2. In this case, however, primer gMLP7.2 with the sequence

CAA CAA CGC TAT gAg AAA gAC ACC

corresponding to SEQ ID No. 15 and gMLP8.2 with the sequence

ggA gCT AgA gAg AAT gAC AgC TgC

corresponding to SEQ ID No. 16 were used under the following PCR conditions:

1. 94° C. for 4 minutes

Then, a total of 36 cycles begin with

2. 94° C. for 45 seconds

3. 60° C. for 45 seconds

4. 72° C. for 1 minute

In connection with the 36 cycles, then:

5. 72° C. for 5 minutes.

A portion of the PCR products obtained was separated in an agarose gel and analyzed to determine its correct size. In the case of correct sizes, which was generally the case, another portion was digested overnight at 37° C. with restriction enzyme Cvi RI and separated in an agarose gel again the next morning. With respect to the incubation, it is to be noted that an incubation of the restriction batch had also been able to take place for 30 to 60 minutes at room temperature. The incubation overnight took place only for the sake of caution.

3.4 Detection of Mutations in Exon 2 or Exon 4 Using Allele-specific Oligonucleotides

In addition to the execution of restriction analyses, probes (so-called allele-specific oligonucleotides, ASO) can also be produced in the discovery of variants or mutations disclosed herein in exon 2 and exon 4 of the genomic human MLP-sequence or the corresponding cDNA.

For the detection of the variant or mutation in exon 2, in this case a probe with the following sequence can be used, which corresponds to SEQ ID No. 4:

AGA TGC CAA ACC GGG GCG

To detect the wild-type-form of exon 2, a probe with the following sequence can be used, which corresponds to SEQ ID No. 6:

AGA TGC CAA ACT GGG GCG

The melting temperature during detection of mutation with use of the sequence according to SEQ ID No. 4 is 60° C., and the melting temperature during detection of the wild type using the sequence according to SEQ ID No. 6 is about 58° C.

For the detection of the variant or mutation in exon 4, in this case, a probe with the following sequence can be used, which corresponds to SEQ ID No. 5:

TTC ACT GCG AAT TTT GGA

For the detection of the wild-type-form of exon 4, a probe with the following sequence can be used, which corresponds to SEQ ID No. 7:

TTC ACT GCA AAG TTT GGA

The melting temperature during detection of the mutation with use of the sequence according to SEQ ID No. 5 is 52° C., and the melting temperature during detection of the wild type with use of the sequence according to SEQ ID No. 7 is about 50° C.

With the indicated primers, exons 2 and 4 of all vehicles of the mutation(s) that were determined by restriction analysis and probe analysis were also sequenced with use of standard techniques. In all cases, the sequence analysis confirmed the result of the restriction analysis and the probe analysis. Thus, the sequencing of exon 2 or 4 or the corresponding cDNA or the corresponding cDNA of the genomic human MLP-gene or the genomic MLP-gene, particularly with use of the probes described herein, can also be used as a process according to the invention for detection of myocardial diseases and/or screening of myocardial diseases, particularly dilatative cardiomyopathy.

3.5 Structure of the Regulatory Nucleic Acid (Sequence)

The regulatory nucleic acid that is indicated in SEQ ID No. 8 is depicted in FIG. 2 together with the labeled promoter-specific elements. The isolation of this regulatory nucleic acid is possible with use of the above-described primers gPl and gP2, corresponding to SEQ ID No. 9 and SEQ ID No. 10. With use of the two above-mentioned primers, the regulatory nucleic acid sequence or the MLP-promoter (1000 bp) that corresponds in this respect can be amplified.

With reference to FIG. 2, the following can be noted herein:

The last sequence that is underlined on the 3′-end corresponds to the first exon.

The AP-1-sequences are labeled by underlining, whereby at most two false pairs were accepted from the consensus sequence TGAG/CTCA.

CAAT sequences are labeled in boldface, whereby in this connection it should be noted that at the beginning of the promoter, an AP-1 sequence and a CAAT sequence overlap.

TATA boxes are labeled in boldface and by underlining.

The disclosure of the various bibliographic references that are cited herein is fully integrated by reference.

The features of the invention that are disclosed in the above description, the claims and the drawing can be essential both individually and in any combinations for the implementation of the invention in its various embodiments.

Claims

1. Nucleic acid that codes for an MLP and comprises a sequence according to SEQ ID NO: 1, whereby the sequence has a mutation at base no. 10 of exon 2 of the translated sequence.

2. Nucleic acid according to claim 1, characterized in that the mutation is an exchange of T for C.

3. Nucleic acid that codes for an MLP and comprises a sequence according to SEQ ID NO:1, whereby the sequence has a mutation at the third position of codon 112 in exon 4 of the translated sequence.

4. Nucleic acid according to claim 3, wherein the mutation is an exchange of A for G.

5. Nucleic acid according to one of the preceding claims, wherein it comprises only the sequences that code for an amino acid sequence.

6. Nucleic acid that codes for an MLP and comprises the sequence of position 58 to 639 of SEQ ID NO: 1, wherein a mutation is present at position 67, particularly a mutation from T to C.

7. Nucleic acid that codes for an MLP and comprises the sequence of position 58 to 639 of SEQ ID NO: 1, wherein at position 393, a mutation is present, particularly a mutation of A to G.

8. Nucleic acid that codes for MLP, wherein the sequence without the degeneration of the genetic code would correspond to a nucleic acid according to one of claims 1 to 7.

9. Nucleic acid that codes for MLP, wherein the nucleic acid hybridizes with a nucleic acid according to one of claims 1 to 8.

10. Amino acid sequence, coded by a nucleic acid according to one of claims 1 to 9 or portions thereof, particularly the sequences or portions thereof that carry mutation(s).

11. MLP that comprises an amino acid sequence that is coded by a nucleic acid according to one of claims 1 to 9 or a portion thereof or an amino acid sequence according to claim 10.

12. Probe for a nucleic acid according to one of claims 1 to 9, particularly for detection or amplification thereof, comprising the sequence of SEQ ID NO: 4.

13. Probe for a nucleic acid according to one of claims 1 to 9, particularly for detection or amplification thereof, comprising the sequences according to SEQ ID NO: 5.

14. Use of a nucleic acid according to one of claims 1 to 9 and/or a probe according to claim 12 or 13 for the diagnosis of and/or screening of myocardial diseases, particularly dilatative cardiomyopathy.

15. Process for detecting and/or screening myocardial diseases, particularly dilatative cardiomyopathy, wherein a nucleic acid according to one of claims 1 to 9 and/or a probe according to claim 12 or 13 is used.

16. Process for detecting and/or screening myocardial diseases, particularly dilatative cardiomyopathy, particularly according to claim 15, wherein
a probe that comprises a sequence that codes for MLP is digested with a restriction enzyme, and
the presence of a mutated sequence that codes for MLP is detected by the occurrence of a restriction enzyme-digestion pattern that is altered relative to the restriction enzyme-digestion pattern of a non-mutated sequence that codes for MLP.

17. Process according to claim 16, wherein the sequence that codes for MLP is amplified before digestion using PCR.

18. Process according to claim 16 or 17, wherein the detection or the screening of a nucleic acid is aimed according to one of claims 1 to 9.

19. Process according to claim 17 or 18, wherein the amplification is carried out with a primer, whereby the primer comprises a sequence according to SEQ ID NO: 13 and/or SEQ ID NO: 14.

20. Process according to one of claims 16 to 19, wherein the restriction enzyme-digestion is carried out by Nci I.

21. Process according to claim 20, wherein in the case of a non-mutated sequence, the PCR product a length of about 308 bp and in the case of a mutation at base no. 10 of exon 2 or a mutation at position 67 of the nucleic acid sequence of SEQ ID NO: 1, whereby the mutation occurs in an exchange of T for C, two PCR products with about 205 and about 103 bp.

22. Process according to one of claims 15 to 17, wherein the detection or the screening of a nucleic acid is aimed according to one of claims 3 to 9.

23. Process according to claim 22, wherein the amplification with a primer is carried out, whereby the primer comprises the sequence according to SEQ ID NO: 15 and/or SEQ ID NO: 16.

24. Process according to claim 22 or 23, wherein the restriction enzyme digestion is carried out by Cvi RI.

25. Process according to claim 15, wherein a hybridization of a sample that is to be studied with a probe that comprises a sequence according to SEQ ID NO: 4 is carried out for the detection or screening of a nucleic acid sequence according to one of claims 1 to 9.

26. Process according to claim 25, wherein the hybridization is carried out at 60° C.

27. Process according to claim 15, wherein a hybridization of a sample that is to be studied with a probe that comprises a sequence according to SEQ ID NO: 5 is carried out for detecting or screening a nucleic acid sequence.

28. Process for detecting and/or screening myocardial diseases, particularly dilatative cardiomyopathy, wherein an amino acid sequence is detected according to claim 10 or an MLP is detected according to claim 11.

29. Process according to claim 28, wherein the amino acid sequence or the MLP is detected using an antibody, preferably a monoclonal antibody.

30. Kit for detecting a nucleic acid according to one of claims 1 to 9, wherein it comprises at least one nucleic acid according to one of claims 1 to 9 and/or at least one probe according to claim 12 or 13.

31. Kit for detecting an MLP according to claim 11 or an amino acid sequence that codes for an MLP according to claim 10 that comprises at least one antibody against an MLP according to claim 11 or against an amino acid sequence according to claim 10.

32. Regulatory nucleic acid that comprises about 500 base pairs upstream from the first base of the first exon of the human genomic sequence that codes for MLP.

33. Regulatory nucleic acid according to claim 32 that comprises about 1000 base pairs upstream from the first base of the first exon of the human genomic sequence that codes for MLP.

34. Regulatory nucleic acid, wherein starting from human genomic DNA with use of two primers in a PCR reaction, the regulatory nucleic acid can be produced, whereby the upstream primer comprises SEQ ID NO: 9 and the downstream primer comprises SEQ ID NO: 10.

35. Regulatory nucleic acid that comprises a sequence according to SEQ ID NO: 8.

36. Regulatory nucleic acid according to one of claims 32 to 35, wherein the regulatory nucleic acid is a promoter.

37. Vector that comprises a regulatory sequence according to one of claims 32 to 36.

38. Vector according to claim 37, wherein the regulatory sequence is contained in the vector pAdCMVssTnI, whereby in this vector, the CMV-promoter is replaced by the regulatory sequence according to one of claims 32 to 36.

39. Vector according to claim 37, wherein it is an adenoviral vector.

40. Vector according to one of claims 37 to 39 that comprises a coding sequence, which is under the control of the regulatory nucleic acid according to one of claims 32 to 36.

41. Vector according to claim 40, wherein the coding sequence is selected from the group that comprises hsp70 and “slow skeletal troponin I.”

42. Cell that comprises a regulatory nucleic acid according to one of claims 32 to 36 and/or a vector according to one of claims 37 to 40.

43. Cell according to claim 42, wherein the cell is a eukaryotic cell, preferably a mammal cell and preferably a human cell.

44. Medication that comprises a regulatory nucleic acid according to one of claims 32 to 36, a vector according to claim 37 to 41 or a cell according to one of claims 42 or 43.

45. Medication according to claim 44, wherein the medication is used within the framework of gene therapy.

46. Medication according to claim 44 or 45, wherein the medication is one for treatment and/or prevention of cardiovascular diseases, particularly cardiovascular diseases in which a point mutation in a gene results in a clinical picture.

47. Medication according to claim 46, wherein the point mutations are in genes that code for proteins of the sarcomere, dystrophin and cardial actin.

48. Medication according to claim 46 or 47, wherein the cardiovascular disease is selected from the group that comprises hypertrophic cardiomyopathy, long QT syndrome and dilatative cardiomyopathy.


SEQUENCE LISTING (17 sequences)

SEQ ID NO: 1 - DNA, 1273 bp, Homo sapiens
  CDS (58)..(642)
gtcgacctgaacggagtccacacaggcagacttgaccttgaccagatagtcttcaagatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcaaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgaagaggtgcgccgtttctcagattttttgcgagcctaaaacacttgccaagtaatcctgcacagatcgatacctttccccaaatagcctctcctttgtagtcgtacattatgtgtttctcctcagaagtgatcaggtctttactgaatgttagaagaggcctttggaagaaaattatgtaaagtttaatctataacaaatgctttattatttataatgcttggaatgggagaggcaataaataaatgttttagtgctatcttgtatggctctagatcttttctttgagatagaaattttcaaaaacataaagctagttcaaaaaacgagttgcagagcatataataaatttggatgtcaactgagaaaggagtgagaaggaagaaataatgcgcaaaggaaagcagtctttcagaatctgtcagccaagtgtctttctagttactgctaatggagaagaaaacagggggtctgggagaaaatagagaacatgatagcaaaatctaaaaggaaaatcaaaactaataaaattgctgaagagttgatccctttgtcctatcgtggggctttgtaatgttacacatctcgtgaaaactcagaaatgacaataaagcgtggcattgcctctgcaaa

SEQ ID NO: 2 - DNA, 1273 bp, Homo sapiens
gtcgacctgaacggagtccacacaggcagacttgaccttgaccagatagtcttcaagatgccaaaccggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcaaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgaagaggtgcgccgtttctcagattttttgcgagcctaaaacacttgccaagtaatcctgcacagatcgatacctttccccaaatagcctctcctttgtagtcgtacattatgtgtttctcctcagaagtgatcaggtctttactgaatgttagaagaggcctttggaagaaaattatgtaaagtttaatctataacaaatgctttattatttataatgcttggaatgggagaggcaataaataaatgttttagtgctatcttgtatggctctagatcttttctttgagatagaaattttcaaaaacataaagctagttcaaaaaacgagttgcagagcatataataaatttggatgtcaactgagaaaggagtgagaaggaagaaataatgcgcaaaggaaagcagtctttcagaatctgtcagccaagtgtctttctagttactgctaatggagaagaaaacagggggtctgggagaaaatagagaacatgatagcaaaatctaaaaggaaaatcaaaactaataaaattgctgaagagttgatccctttgtcctatcgtggggctttgtaatgttacacatctcgtgaaaactcagaaatgacaataaagcgtggcattgcctctgcaaa

SEQ ID NO: 3 - DNA, 1273 bp, Homo sapiens
gtcgacctgaacggagtccacacaggcagacttgaccttgaccagatagtcttcaagatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgaagaggtgcgccgtttctcagattttttgcgagcctaaaacacttgccaagtaatcctgcacagatcgatacctttccccaaatagcctctcctttgtagtcgtacattatgtgtttctcctcagaagtgatcaggtctttactgaatgttagaagaggcctttggaagaaaattatgtaaagtttaatctataacaaatgctttattatttataatgcttggaatgggagaggcaataaataaatgttttagtgctatcttgtatggctctagatcttttctttgagatagaaattttcaaaaacataaagctagttcaaaaaacgagttgcagagcatataataaatttggatgtcaactgagaaaggagtgagaaggaagaaataatgcgcaaaggaaagcagtctttcagaatctgtcagccaagtgtctttctagttactgctaatggagaagaaaacagggggtctgggagaaaatagagaacatgatagcaaaatctaaaaggaaaatcaaaactaataaaattgctgaagagttgatccctttgtcctatcgtggggctttgtaatgttacacatctcgtgaaaactcagaaatgacaataaagcgtggcattgcctctgcaaa

SEQ ID NO: 4 - DNA, 18 bp, Artificial Sequence
  Description of Artificial Sequence Primer or probe
agatgccaaaccggggcg

SEQ ID NO: 5 - DNA, 18 bp, Artificial Sequence
  Description of Artificial Sequence Primer or probe
ttcactgcgaagtttgga

SEQ ID NO: 6 - DNA, 18 bp, Artificial Sequence
  Description of Artificial Sequence Primer or probe
agatgccaaactggggcg

SEQ ID NO: 7 - DNA, 18 bp, Artificial Sequence
  Description of Artificial Sequence Primer or probe
ttcactgcaaagtttgga

SEQ ID NO: 8 - DNA, 1037 bp, Homo sapiens
atctgatgtgaaggctggatcatgaacctcccggttgggttctacatgtaccttacctcaacatccgggtcaacatgagtggaaggggcttggtgcctcagtttcctcatctataacatgaagaattggtcttcacatctctgaacctcttgtcagcttttcaatgccataatttatatcagtccctggtgcttgatcaaatttaaacggtgtgaattccagacaaagaaaaattagacctccaaagaaaatatgggtctacagagacatatcaagatgttataaatataatgtgatgatgataaatatgcactgctgtatcaaattcttaaatagaaaaggataataacaaagtaaatcaaaactttttagagaaaacttcgtttcctaggatagatggagtgacaggtcccaagtgatgtgccaaccttttttctcccaagcttctttgaagagattatctaagtatcgtttcagacacccatcacccttatttttagctctgagttctcttcacgagtgtggtgacctgtggctctaagttccagaggctcccagatatggcacatgactcatttgcaggtaccctgcaatgatttccaggaaggtcagtggggtgggggcctggaaaaatgatttttgatgttaaacccttagctcttgttcctgttccaaccacttgatcagaaatgacacacttcataaattgatcccttggtgccataagcaattggcagtgcaaatagaacccaagtgattcggttctccaagaggctcacagctggctggggggttgggggagacaaaactcagggctttggtcacagtctattttcagcccctgatagcagttgtgttactcacgtctcattcaccctctcccttggcctccctcctgcccttcccccagcaggccaaggctggtgacagccttcatatatttaaagaggacaagagcccctcagactcagttgagctgaacggagtccacacaggcaggtgagtggagcaaggcagacatttgagctggaggtggg

SEQ ID NO: 9 - DNA, 18 bp, Artificial Sequence
  Description of Artificial Sequence Primer or probe
atctgatgtgaaggctgg

SEQ ID NO: 10 - DNA, 18 bp, Artificial Sequence
  Description of Artificial Sequence Primer or probe
cctgcctgtgtggactcc

SEQ ID NO: 11 - DNA, 20 bp, Artificial Sequence
  Description of Artificial Sequence Primer or probe
ggcagacttgaccttgacca

SEQ ID NO: 12 - DNA, 20 bp, Artificial Sequence
  Description of Artificial Sequence Primer or probe
gaggtgcgccgtttctcaga

SEQ ID NO: 13 - DNA, 23 bp, Artificial Sequence
  Description of Artificial Sequence Primer or probe
actcttagagattggttcactcc

SEQ ID NO: 14 - DNA, 23 bp, Artificial Sequence
  Description of Artificial Sequence Primer or probe
accacactatgagaaccactggc

SEQ ID NO: 15 - DNA, 24 bp, Artificial Sequence
  Description of Artificial Sequence Primer or probe
caacaacgctatgagaaagacacc

SEQ ID NO: 16 - DNA, 24 bp, Artificial Sequence
  Description of Artificial Sequence Primer or probe
ggagctagagagaatgacagctgc

SEQ ID NO: 17 - protein, 194 aa, Homo sapiens
MPNWGGGAKCGACEKTVYHAEEIQCNGRSFHKTCFHCMACRKALDSTTVAAHESEIYCKVCYGRRYGPKGIGYGQGAGCLSTDTGEHLGLQFQQSPKPARSVTTSNPSKFTAKFGESEKCPRCGKSVYAAEKVMGGGKPWHKTCFRCAICGKSLESTNVTDKDGELYCKVCYAKNFGPTGIGFGGLTQQVEKKE